(19) United States
(12) Patent Application Publication
(10) Pub. No.: US 2007/0264315 A1
(43) Pub. Date: Nov. 15, 2007

(54) PASTE USABLE AS A DRESSING ON THE ORAL MUCOSAE OR THE SKIN

(75) Inventors: Marguerite FOURNIE, ANDERNOS LES BAINS (FR); Patrick LESAGE, SAINT MALO (FR)

Correspondence Address: LUCAS & MERCANTI, LLP, 475 PARK AVENUE SOUTH, 15TH FLOOR, NEW YORK, NY, 10016, US

(73) Assignee: PRODUITS DENTAIRES PIERRE ROLLAND, MERIGNAC (FR)

(21) Appl. No.: 11/736,785

(22) Filed: Apr. 18, 2007

(30) Foreign Application Priority Data
May 10, 2006 (FR) 0651678
Mar. 16, 2007 (FR) PCT/FR07/50938

(51) Int. Cl. A61L 15/08; A61K 33/06

(52) U.S. Cl. 424/445

(57) ABSTRACT

The present invention relates to a biocompatible paste containing an aqueous excipient which can be used to form a dressing by the application of said paste to the oral mucosae or the skin, containing the following as essential constituents:
natural kaolin containing at least 80% by weight, preferably at least 95% by weight, of kaolinite;
a humectant selected from the group consisting of propylene glycol, glycerol, polyethylene glycol, sorbitol and mixtures thereof; and
a hydrogelling agent forming a hydrogel with part of the water present in said excipient, which is selected from the group consisting of anhydrous colloidal silica, cellulose, carboxymethyl cellulose, guar gum, xanthan gum and mixtures thereof.

It further relates to a method of stopping the oral mucosae or the skin from bleeding, by applying said paste in efficient amount to form a dressing, especially a hemostatic dressing.

The invention relates to a biocompatible paste containing an aqueous excipient which can be used as a dressing, particularly as a hemostatic dressing on the oral mucosae or the skin.

Self-adhesive dressings consisting of gauze and adhesive plaster, for stopping a wound from bleeding, are known.

Liquid dressings, which are applied to the wound and solidify rapidly, drying to form a protective film, are also known.

These devices are intended to remain in place until the wound has healed, and must then be removed, which can present an esthetic problem; they are removed either by detaching the adhesive part or by rubbing the solidified dressing, which can be painful.

Furthermore, these devices for stopping bleeding are not suitable for the dental sector, since they do not adhere or do not solidify in the moist environment of the mouth: the adhesive of the adhesive dressing does not stick to the mucosae and the liquid gel does not solidify in this moist environment of the mouth.

Thus, when administering dental care, particularly descaling, the treatment of caries and the taking of impressions, which can cause bleeding that prevents the practitioner from seeing the treatment site, not only must the blood be voided, but also its flow must be stopped to enable the treatment to continue and also to enable the patient to leave without bleeding.

In this sector, pads consisting of an absorbent material, generally hydrophilic cotton, have hitherto been used to absorb the blood. This type of absorbent pad has to be pressed onto the wound in order to stop the bleeding by compression and in order to be held in place, which can be painful for the patient.

Materials in the form of a paste which are intended to be used as insertion materials for widening the gingival sulcus, while avoiding bleeding during dental treatment, are already known. Such materials are described more particularly in patent EP 0 477 244, which describes insertion materials that can be used for widening the gingival sulcus substantially without bleeding or weeping, especially when taking an impression for a dental prosthesis.

A well-defined plastic viscosity and a well-defined yield point are sought for such materials, and a kaolinic clay can be chosen in particular as one of the constituents of these materials, being present in said materials in a proportion of between 65 and 70% by weight.

However, no attempt is made to apply materials of this type as dressings, which notably requires very particular adhesive properties.

Other materials likewise intended for achieving gingival retraction are also described in international patent application WO 2005/007095, which again describes materials for avoiding gingival retraction and, inter alia, proposes materials that contain for this purpose a clay such as kaolin and additional constituents known for their hemostatic or coagulant effect.

However, here again, as in the previous document, no attempt is made to achieve an adhesive effect on the gums and to form a true protective dressing.

Furthermore, international patent application WO 96/25915 describes a viscous solution for controlling gum bleeding during a dental intervention. The essential constituents present in such a composition are a hemostatic agent and an agent for reducing the acidity of the latter, which can be an inorganic filler or a high-molecular polyol.

American U.S. Pat. No. 6,652,840B1 describes compositions for controlling gum bleeding and improving gum healing.

These compositions preferably comprise aluminum chloride, ferric sulfate, oxidized regenerated cellulose, aluminum ammonium sulfate, gelatin and a solvent. Once again, however, even though it has hemostatic and healing properties by virtue of its constituents, this composition does not allow the formation of a protective dressing on the gum.

The inventors of the present invention have now discovered novel pasty compositions in which kaolin is associated with various other well-defined substances and which, after application to the oral mucosae, make it possible to obtain a true dressing for stopping bleeding during dental interventions.

They have also discovered that these same compositions can be used for application to the skin, thereby allowing the formation of a true dressing from a paste.

Thus the invention aims to overcome the disadvantages of the devices of the prior art and makes it possible to stop bleeding by proposing a paste which, when applied to the wound, stops the bleeding by a simple barrier effect, stays in place without the need to apply pressure or adhesive to the wound, even in a moist environment or on non-horizontal walls, and is removed without causing pain to the patient.

More precisely, according to a first feature, the invention relates to a biocompatible paste containing an aqueous excipient which can be used to form a dressing by the application of said paste to the oral mucosae or the skin, the essential constituents of said paste being as follows:

natural kaolin containing at least 80% by weight, preferably at least 95% by weight, of kaolinite;

a humectant selected from the group consisting of propylene glycol, glycerol, polyethylene glycol, sorbitol and mixtures thereof; and

a hydrogelling agent forming a hydrogel with part of the water present in said excipient, which is selected from the group consisting of anhydrous colloidal silica, cellulose, carboxymethyl cellulose, guar gum, xanthan gum and mixtures thereof.

According to a second feature, the invention relates to the use of the above paste as a dressing or for the preparation of a dressing, said dressing being intended for application to the oral mucosae or the skin.

According to a third feature, the invention relates to a method of treatment for stopping the oral mucosae or the skin from bleeding, which consists in applying the paste forming the subject of the first feature of the invention to the appropriate part of the body, in efficient amount to form a dressing.

The invention will be better understood and other characteristics and advantages thereof will become more clearly apparent from the following explanatory description referring to the Figures, in which:

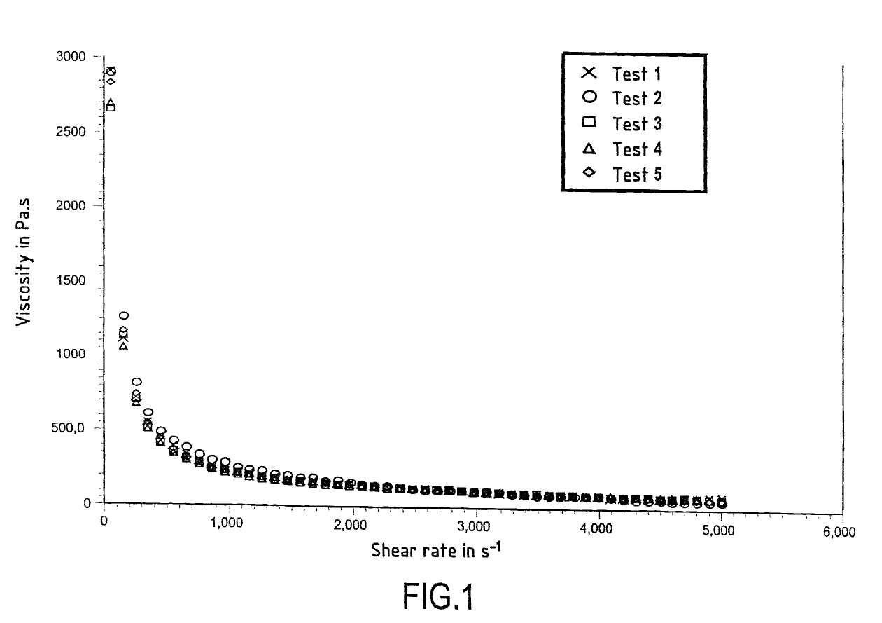

FIG. 1 shows the rheofluidifying behavior of the paste obtained according to the invention in Example 1,

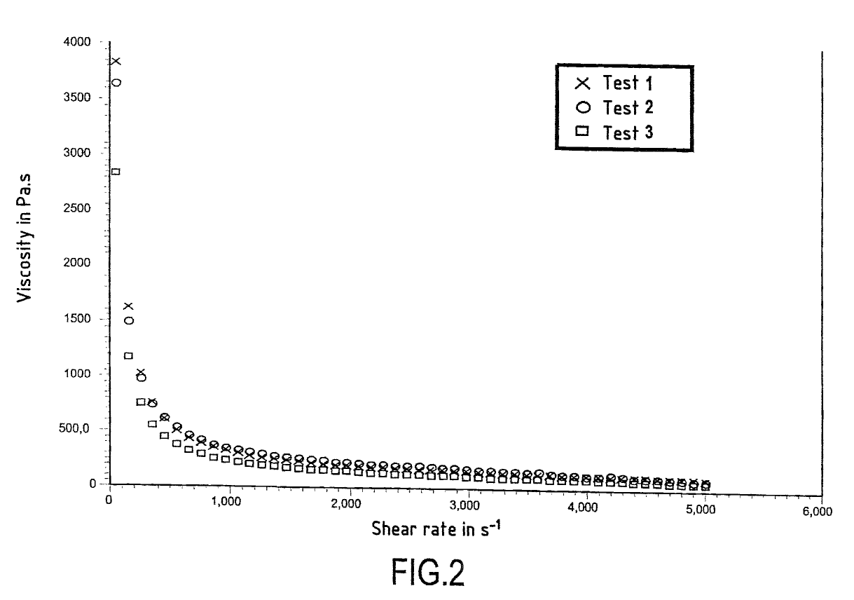

FIG. 2 shows the rheofluidifying behavior of the paste obtained according to the invention in Example 1, after a centrifugation treatment,

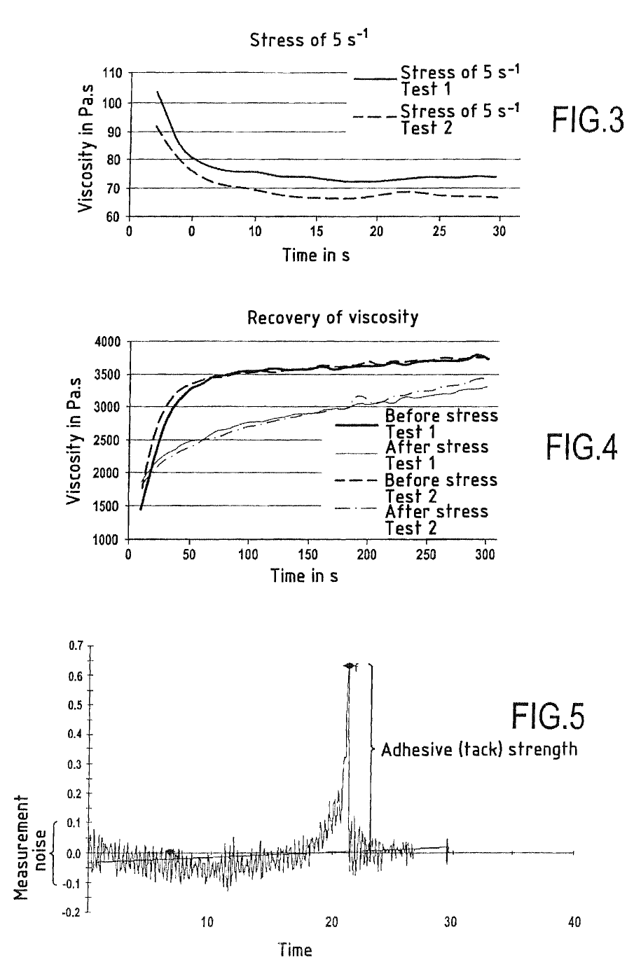

FIG. 3 shows the change in viscosity of the paste obtained according to the invention in Example 1 as a function of time, at a stress of 5 s⁻¹,

FIG. 4 shows the thixotropic behavior of the paste obtained according to the invention in Example 1, and

FIG. 5 is the curve showing measurement of the adhesive strength (also called tack strength) of the paste obtained according to the invention in Example 1.

In the different features of the invention, the paste of the invention is a biocompatible paste and the water used for its preparation is advantageously prepurified water as conventionally used in products that are to be brought into contact with a wound.

As essential components, this paste contains natural kaolin, a humectant selected from the group comprising propylene glycol, glycerol, polyethylene glycol, sorbitol and mixtures thereof, and a hydrogelling agent selected from the group consisting of an anhydrous colloidal silica, a cellulose, carboxymethyl cellulose, guar gum, xanthan gum and mixtures thereof.

In the presence of water introduced in a sufficient amount to substantially saturate the kaolin and form a hydrogel by virtue of the presence of the hydrogelling agent, these three essential constituents make it possible to obtain a composition in the form of a paste whose rheological properties and adhesive (tack) properties are suitable for achieving the effect of a dressing on the oral mucosae or the skin when this paste is applied.

Those skilled in the art will of course understand that the proportions of the essential constituents and that of the water present in the composition can vary according to the nature of the two constituents represented by the hydrogelling agent and the hydrating agent.

The kaolin contains at least 80% by weight and advantageously at least 95% by weight of kaolinite and acts as a fluid absorber in the paste of the invention. It has the general properties of the clays known in medicine and cosmetics, but, in particular, in combination with the hydrogelling agent and the humectant, it gives the paste of the invention the desired texture and theological behavior.

The (preferably purified) water is introduced in a sufficient amount to give a paste of the desired viscosity, texture and appearance.

The humectant makes it possible on the one hand to obtain the visual appearance of the product and to prevent the paste from drying out during storage.

In particular, in cooperation with the other two essential constituents of the invention represented by the hydrogelling agent and the kaolin, the humectant makes it possible to obtain the desired texture and rheological behavior of the paste. The hydrogelling agent additionally gives the paste properties of adhesion to the skin or the oral mucosae.

The hydrogelling agent is preferably an anhydrous colloidal silica and particularly preferably one with a specific surface area in the order of 200 m²/g. An example of such an anhydrous colloidal silica is the product marketed under the mark Aérosil® 200 by Degussa.

In one particularly advantageous variant, the humectant is propylene glycol.

As explained above, the proportions of the essential constituents vary according to their nature.

However, in one particularly advantageous variant of the invention, in the case where the hydrogelling agent is anhydrous colloidal silica, the paste comprises the following proportions by weight of essential constituents and water:
kaolin: between 35 and 55%,
propylene glycol: between 2 and 4%,
anhydrous colloidal silica: between 3 and 7%,
water: between 35 and 46%.

By virtue of the presence of the kaolin, the paste of the invention as defined above already possesses valuable hemostatic properties.

However, it will often be advantageous to add to this paste an astringent, which contributes to stopping the bleeding more rapidly through its tissue retracting action, and/or a vasoconstrictor, which also contributes to stopping the bleeding more rapidly through its constricting action on the blood vessels.

This astringent will advantageously be selected from the group comprising iron or aluminum chlorides and sulfates, potassium aluminum sulfate and mixtures thereof.

Preferably, the astringent will be aluminum chloride and will be present in the composition in a concentration advantageously of between 5 and 25% by weight, preferably of about 15% by weight, based on the total weight of the paste.

Finally, a vasoconstrictor, e.g. adrenaline, may also be introduced into the paste of the invention.

As explained above, the value of the compositions of the invention is their ability to form a dressing when applied to the skin or the oral mucosae. This imposes very specific rheological and adhesive properties on the paste, which are advantageously as follows:
the paste has a rheofluidifying behavior at 23° C.,
it has a viscosity at rest of between 3000 and 4500 Pa·s, preferably of between 3500 and 4000 Pa·s, measured at 23° C.,
it has a thixotropic behavior, and
it has an adhesive strength of between 0.3 and 0.7 N, preferably of between 0.5 and 0.6 N, measured at 23° C. and ambient humidity.

This rheofluidifying behavior is characterized by a yield stress of between 110 and 140 Pa, measured at 23° C., and a viscosity of between 60 and 90 Pa·s, measured at a shear rate of 5 s⁻¹ and a temperature of 23° C.

Thus the paste of the invention preferably has a rheofluidifying behavior at 23° C. This means that the viscosity of the paste according to the invention drops when a stress is applied. Thus, at rest, the paste of the invention has a viscosity of between 3000 and 4500 Pa·s, measured at 23° C. This viscosity enables it to remain in place, even on non-horizontal substrates, without flowing.

However, when applying a low stress, such as a stress corresponding to a mixing action with a spatula, the viscosity of the paste drops, enabling it to be applied to and smoothed over the skin or the oral mucosae without any effort.

This behavior also enables it to be applied to the application site with a syringe by simply using manual force, without exerting excessive pressure.

This constitutes an advantage for the compositions of the invention, which will advantageously be packaged in syringes, particularly those with disposable tips, especially for use by dentists.

The paste of the invention has a thixotropic behavior; in other words, even after manual agitation or the application of pressure to eject it from the syringe, it recovers its original viscosity once the agitation or the application of pressure has stopped.

Furthermore, the paste of the invention adheres to the skin and also to the oral mucosae, which are a moist environment at 37° C., without the application of pressure to hold it in place.

This adhesive property corresponds to an adhesive (tack) strength of between 0.3 and 0.7 N, measured at 23° C. and ambient humidity.

When measured at 37° C. and 90% relative humidity in order to simulate the humidity and temperature conditions of the mouth, the adhesive properties of the paste of the invention remain excellent.

Preferably, the adhesive (tack) strength of the paste of the invention is between 0.5 and 0.6 N.

As regards the rheofluidifying behavior of the paste, this is characterized by a yield stress of between 110 and 140 Pa, measured at 23° C., and a viscosity of between 60 and 90 Pa·s, measured at a shear rate of 5 s⁻¹ and a temperature of 23° C.

Thus, by virtue of its theological behavior, advantageously enabling it to flow easily under a low stress, the paste of the invention can be packaged in a syringe, allowing the product to be applied in a precise and localized manner and to be kept aseptic.

Of course, the paste of the invention is biologically compatible.

The paste of the invention can be used to stop the skin from bleeding and also to stop the oral mucosae from bleeding, so it is particularly suitable for use by dentists when they are operating. In fact, the paste of the invention may be applied with a syringe or with a spatula, without exerting pressure.

Once in place, its adhesive properties are such that it adheres to the oral mucosae and stops the bleeding without having to be held by the application of pressure.

Finally, the paste of the invention advantageously contains a colorant. The presence of the colorant is of particular value in dental applications because it will enable the dentist to locate the wound more easily during the intervention.

It is for this reason that a blue colorant will preferably be chosen in dental applications.

The paste of the invention will also advantageously comprise a flavoring.

Both the colorant and the flavoring will advantageously be selected from food-grade products.

Of course, those skilled in the art will readily understand that the amounts and concentrations of the colorants and/or flavorings, and in general of the products used in the pastes of the invention, will have to be determined so as not to modify the rheological and adhesive properties of the composition.

One particularly preferred paste according to the invention comprises between 0.03 and 0.1% of food-grade blue colorant and/or between 0.5 and 2% of food-grade flavoring, in percentages by weight based on the total weight of the paste.

Preferably, if present, the colorant must be of food grade and present in the paste of the invention in a percentage by weight of 0.05%, based on the total weight of the paste.

The use of a blue colorant is particularly advantageous, especially on the mucosae.

If present in the paste according to the invention, the food-grade flavoring is preferably present in a concentration of 1% by weight, based on the total weight of the paste.

As explained above, the paste of the invention preferably contains a blue colorant so that the dentist can precisely locate the application site of the paste. Once bleeding has stopped, the paste of the invention is removed simply by rinsing with water, no rubbing being necessary. Here again, the blue coloration will enable the dentist to assess visually that all the paste has been removed.

He will then be able to continue treatment as required.

As indicated above, by virtue of their composition and their ability to adhere to the skin or the oral mucosae, the pastes of the invention can advantageously be used as a dressing and very particularly as a hemostatic dressing.

To provide a better understanding of the invention, several embodiments thereof will now be described by way of purely illustrative and non-limiting Examples.

### EXAMPLE 1

A paste according to the preferred embodiment of the invention is obtained by the following process. This process is carried out at room temperature.

38 g of water are weighed out.

10 g of this water are taken and 0.05 g of food-grade blue colorant is dissolved therein to give a first solution.

15 g of aluminum chloride are then dissolved in the remainder of the water.

When the aluminum chloride has completely dissolved, 3 g of propylene glycol and 1 g of flood-grade strawberry flavoring are added.

This solution is homogenized to give a second solution.

The first solution containing the food-grade blue colorant is then introduced into the second solution and the whole is homogenized.

The pH of the resulting solution is 2.

4.05 g of Aérosil® 200 are added to this solution and the product is mixed to give a gel.

38.9 g of commercial kaolin containing at least 95% by weight of kaolinite are added gradually to this gel.

When all the kaolin has been incorporated, mixing is continued in order to homogenize the product.

In this Example, which is the particularly preferred embodiment of the invention, the aluminum chloride is used as an astringent,

Rheological Behavior

The theological behavior of the paste obtained in this Example was measured using a flow test in which the sample of paste is stressed at a given rate of strain (shear rate) and the corresponding stress is measured for increasing rate of strain values.

The following procedure is used:

Equipment: AR 1000 rheometer from TA Instruments

Data processing software: Rheology Advantage Data Analysis V5.1.42

Type of test: flow with measurements at equilibrium, the following applying to each point:

validation by three concordant samplings (tolerance: 5%)

sampling period: 10 s

maximum measurement time per point: 1 minute

Number of points: 50, with linear distribution

Range of shear rates: 0.05-5 s⁻¹

Temperature: 23° C., controlled by Pelletier Plate (precision: 0.1° C.)

Measuring geometry: 25 mm anodized aluminum plate

Air gap: 1200 μm

Product deposited with a spoon

Manual descent to 1500 μm

Automatic descent to 1250 μm

Cleaning of surplus sample

Automatic descent to 1200 μm

The results obtained are presented in FIG. 1, which shows the change in the viscosity of the paste as a function of the shear stress. This curve clearly shows the rheofluidifying behavior of the product, i.e. that its viscosity decreases with increasing shear rate.

This drop in viscosity is very marked up to 2 s⁻¹, after which a kind of plateau is observed, the viscosity decreasing at a much slower rate.

The Figure also shows the presence of a vertical asymptote for shear rates close to zero. This means that the viscosity is infinite for a zero shear rate. In other words, the paste has a very high flow resistance when at rest. This property enables the paste to “sit” and not to flow when it is subjected to shear stresses below a value known as the yield stress.

The behavior of the product can be represented by the Hershel-Bulkley equation:

μ=σₛ/γ⁰+β(γ⁰)ⁿ⁻¹

where σₛ (yield stress), β and n are constant.

The yield stresses over 5 tests were obtained by performing an extrapolation calculation centered more on the low shear rates. As regards the viscosity at 5 s⁻¹, this was measured using the processing software.

The yield stress values and the viscosity values obtained, measured at 5 s⁻¹, are collated in Table I below:

TABLE I

| | Test | Test | Test | Test | Test | | Standard |
|---|---|---|---|---|---|---|---|
| | 1 | 2 | 3 | 4 | 5 | Mean | deviation |
| Yield stress (Pa) | 134 | 113 | 113 | 122 | 124 | 121 | 9 |
| Viscosity at 5 s⁻¹ | 87 | 48 | 62 | 72 | 60 | 66 | 15 |
| (Pa · s) | | | | | | | |

To demonstrate the stability of this rheological behavior and evaluate the effect of sedimentation of the paste, some of the paste obtained in Example 1 was centrifuged and the bottom part of the centrifuged sample was collected. This bottom part is subjected to the same flow test as described above.

The curves shown in FIG. 2 are obtained for two tests.

FIG. 2 shows that the behavior of the paste of the invention after centrifugation is again a rheofluidifying behavior.

If the curves obtained in FIGS. 1 and 2 are compared, it is seen that the paste seems to be slightly thicker up to 4 s⁻¹, after centrifugation. Beyond this stress value, the measured viscosity values are equivalent and the difference is no longer pronounced.

Table II below collates the yield stress values and the corresponding viscosities obtained in the paste of the invention after centrifugation.

TABLE II

| | Test 1 | Test 2 | Mean |
|---|---|---|---|
| Yield stress (Pa) | 164 | 159 | 161 |
| Viscosity at 5 s⁻¹ (Pa · s) | 99 | 80 | 89 |

It is seen that sedimentation has a slight influence on the paste, which is found to be slightly thickened.

This results in a slightly higher yield stress in the order of 160 Pa instead of 120, but there is no distinct variation of the plateau viscosity, which remains of the same order of magnitude.

Thixotropic Behavior

The thixotropic behavior of the paste obtained in this Example was also demonstrated.

This thixotropic behavior was evaluated on the uncentrifuged sample obtained in Example 1.

The thixotropic character of the paste of the invention, i.e. its capacity to recover its initial viscosity (viscosity at rest) after being stressed, was demonstrated using the following test carried out at 23° C.:

shear at a very low shear rate (0.05 s⁻¹) for 5 minutes after sampling every 10 seconds

shear at 5 s⁻¹ for 30 seconds with sampling every 2 seconds

repeat of step 1

measuring geometry: 25 mm aluminum plate

air gap: 1200 μm

This test makes it possible to visualize the recovery of the viscosity after shear by comparing the viscosity obtained at equilibrium during the first step with that measured during the third step.

The results obtained are presented in FIGS. 3 and 4, which show the change in the viscosity over time, before and after stress.

FIG. 3 shows the change in the viscosity during the second step of the test, i.e. at a “high” shear of 5 s⁻¹. It is seen from FIG. 3 that crazing is very distinct and that a plateau viscosity is rapidly reached.

FIG. 4 shows the measurements made during steps 1 and 3 at a “low” shear (0.05 s⁻¹), which simulate rest. The first step makes it possible to evaluate the viscosity at rest, which is then between 3500 and 4000 Pa·s.

It is seen in the third step that the fluid tends to recover its initial viscosity after stress, this reversibility being characteristic of thixotropy.

Two types of test were carried out to demonstrate the adhesive properties of the paste obtained in the Example of the invention.

Firstly, a test was carried out at 23° C. and ambient humidity.

These tests are based on FINAT standard test method no. 9 with the following exceptions:

ambient humidity

sample adhesive coated on fixed substrate (aluminum plate)

contact area: 25 mm×25 mm

sensor: 5 N+0.025 N

the paper used has a basis weight of 80 g/m².

These tests correspond to loop tack tests.

The loop tack test consists in bringing a strip of paper into contact with a sample and measuring the force required to detach the paper, i.e. the tack strength. The contact area is 625 mm² (i.e. 25 mm×25 mm).

This test does not involve push contact; in other words, the loop is simply placed on the surface to be studied, in contrast to the needle tack tests, where a maintenance pressure is applied during contact.

Although the name of this test refers to the concept of tack, it actually measures stickiness, a term employed e.g. to describe jam or soft chocolate.

The measured adhesive (tack) strengths (in N) are collated in Table III below:

TABLE III

| | | | | Test | Test | | Standard |
|---|---|---|---|---|---|---|---|
| | Test 1 | Test 2 | Test 3 | 4 | 5 | Mean | deviation |
| Paste of | 0.6 | 0.6 | 0.5 | 0.5 | 0.4 | 0.5 | 0.1 |
| Example 1 | | | | | | | |

The curve representing the behavior of the paste obtained in Example 1 is shown in FIG. 5.

In the case of the paste of the invention, the “loop tack” test does not enable the adhesive strength to be measured because the measured value is of the same order as the measurement noise. It can therefore be said that, in the case of simple contact, without pressure, the paste of the invention exhibits adhesive properties. The rupture pattern observed with the paste of the invention confirms this conclusion. In fact, a deposit of paste of the invention remains on the loop after detachment. It will be noted that the measured value of the tack strength cannot be associated with this inevitable excess weight of the loop, since the curve returns to zero (measurement noise after the detachment peak).

Furthermore, it should be emphasized that there is a correlation between the cohesion of the paste of the invention and its adhesion to a surface.

The tack strength is defined as the force required to separate a material that possesses instantaneous adhesion, i.e. contact adhesion, from a surface that is to be stuck to said material. Now, the rupture is cohesive, meaning that the adhesion of the paste of the invention is greater than the cohesion. The term stickiness is used under these conditions.

However, this low cohesion in no way detracts from the intended application, since the paste of the invention is used in thin layers on the skin or the gum.

The yield stress is then sufficiently large to prevent the bulk of the product from flowing under the effect of gravity or flowing blood.

“Loop tack” tests were also carried out in a controlled atmosphere, simulating the conditions found in the mouth.

These tests are also based on FINAT standard test method no. 9 with the following values:

temperature: 37° C.

humidity: 90%

adhesive (sample) coated on the borosilicate glass sample holder of the system

contact area: 25 mm×40 mm

sensor: 5 N±0.025 N

The paper used has a basis weight of 80 g/m².

The tack strengths measured on the sample paste are collated in Table IV below:

TABLE IV

| | | | | Test | Test | | Standard |
|---|---|---|---|---|---|---|---|
| | Test 1 | Test 2 | Test 3 | 4 | 5 | Mean | deviation |
| Paste of | 0.3 | 0.1 | 0.1 | 0.1 | 0.2 | 0.2 | 0.1 |
| Example 1 | | | | | | | |

The values obtained under these conditions differ from those obtained at 23° C. and ambient humidity.

In fact, the high humidity of the environment causes the paste of the invention to absorb water and become filled with water. However, the same type of rupture continues to be observed, meaning that, under these conditions, the paste of the invention still makes it possible to obtain adhesion on the gum without applying pressure.

Furthermore, if it is considered that, at the moment of coating on the wound, the ambient humidity of the mouth ultimately has only a small influence on the behavior of the paste, since it does not have the time to acclimatize to the humidity conditions of the mouth during this short phase.

Measurement of the Contact Angles

The adhesive properties of the paste of the invention were also demonstrated by contact angle measurements.

The object of these measurements is to measure the contact angles formed by drops of reference liquid of controlled volume (in this case two microliters) deposited on the surface of a given substrate (in this case the paste of Example 1). The angles are measured on the right and left of the drop for better precision, two seconds after the drop has been deposited.

The reference liquids used are water and isotonic solution, which gives results similar to those obtained in the case of the saliva and blood bathing the gum.

The paste of Example 1 was coated on a microscope slide until a satisfactory surface evenness was obtained. About 1 g of this paste was used to give a film having a thickness of about 1 mm and a surface area of 10 cm², corresponding to a basis weight of about 1 kg of paste per m² of slide.

The contact angle is measured on at least 5 samples of the reference liquids with the paste of Example 1.

Measurement of the contact angle makes it possible to visualize the spreading of the reference liquids over the paste obtained in Example 1, i.e. the affinity of these liquids for the paste of Example 1. These measurements give an idea of the affinity of the paste of Example 1 for the liquids in contact with the gum, such as saliva and blood.

In the case of a very weak interaction between the sample and the substrate (paste of Example 1), the measured contact angle will be high, since the sample attempts to minimize its contact area with the substrate (paste of Example 1).

In the opposite case, the sample will spread very well over the substrate (paste of Example 1) and the angle will be very small.

The values of the angles measured using the paste of the invention obtained in Example 1 as substrate, and drops of water and/or drops of saline solution, are presented in Table V below.

TABLE V

| | | | | | | | Standard |
|---|---|---|---|---|---|---|---|
| | Drop 1 | Drop 2 | Drop 3 | Drop 4 | Drop 5 | Mean | deviation |
| Water | 19–19 | 17.2–21.8 | 20.3–20.1 | 20.9–19.7 | 19.9–21.4 | 20 | 1 |
| Saline | 16.5–14.9 | 17.9–16.4 | 14.4–16.2 | 14.7–14.4 | 13.4–15.8 | 15 | 1 |
| solution | | | | | | | |

It is therefore seen that the drops have a fairly small contact angle, showing that the paste obtained in Example 1 will indeed wet the surface of the gum when it comes into contact therewith.

Tests of Tolerance and Anti-Bleeding Efficacy

Tolerance and biocompatibility tests were performed by applying the paste of the invention obtained in Example 1 to injured hamster cheeks.

These tests showed the perfect tolerance and biocompatibility of the paste of the invention.

They also showed that the paste of the invention obtained in Example 1 makes it possible to stop bleeding by application for about 2 minutes.

As the paste of the invention does not dry out, even several hours after application, it is removed after these 2 minutes by simply washing with water, without the need for rubbing.

### EXAMPLE 2

A process for the production of a paste according to the invention that does not contain aluminum chloride is given below.

This process is carried out at room temperature.

45.55 g of water are weighed out.

10 g of this water are taken and 0.05 g of food-grade blue colorant is dissolved therein to give a first solution.

3 g of propylene glycol and 1 g of food-grade strawberry flavoring are dissolved in the remainder of the water and the solution is homogenized. The first solution containing the colorant is then added to this solution and the whole is homogenized. This addition of acid gives a pH comparable to that of the composition of Example 1.

0.4 g of tartaric acid crystals is added and the whole is mixed and homogenized.

The pH of the resulting solution is 2.

4.05 g of Aérosil 200 are then added and the whole is mixed to give a gel.

46 g of kaolin are added gradually to this gel. When all the kaolin has been incorporated, mixing is continued in order to homogenize the product.

The results in terms of theological behavior, viscosity, thixotropy, contact angles and tolerance are similar to the results obtained with the paste according to Example 1.

It is thus seen that the paste of the invention exhibits a stickiness which allows adhesion without pressure when used as a mechanical dressing on a skin or gum wound; this is a great asset to guaranteeing the painless nature of the operation and the cleanliness of the wound after treatment, since no appreciable pressure will have been exerted on the wound.

Furthermore, the paste of the invention is removed very easily and painlessly.

Of course, the invention is in no way limited to the preferred embodiments, which have been given solely by way of illustration and without implying a limitation.

What is claimed is:

1. A biocompatible paste containing an aqueous excipient, usable to form a dressing by the application of said paste to the oral mucosae or the skin, containing the following as essential constituents:
natural kaolin containing at least 80% by weight of kaolinite;
a humectant selected from the group consisting of propylene glycol, glycerol, polyethylene glycol, sorbitol and mixtures thereof, and
a hydrogelling agent forming a hydrogel with part of the water present in said excipient, which is selected from the group consisting of anhydrous colloidal silica, cellulose, carboxymethyl cellulose, guar gum, xanthan gum and mixtures thereof.

2. The paste according to claim 1, wherein said natural kaolin contains at least 95% by weight of kaolinite.

3. The paste according to claim 1, wherein said hydrogelling agent is anhydrous colloidal silica.

4. The paste according to claim 3, wherein said anhydrous colloidal silica has a specific surface area in the order of 200 m²/g.

5. The paste according to claim 1, wherein said humectant is propylene glycol.

6. The paste according to claim 1, wherein it comprises the following in percentages by weight, based on the total weight of the paste:
kaolin: between 35 and 55%,
propylene glycol: between 2 and 4%,
anhydrous colloidal silica: between 3 and 7%,
water: between 35 and 46%.

7. The paste according to claim 1, wherein it also contains an astringent selected from the group consisting of iron or aluminum chlorides and sulfates, potassium aluminum sulfate and mixtures thereof.

8. The paste according to claim 7, wherein said astringent is aluminum chloride and represents between 5 and 25% by weight, based on the total weight of the paste.

9. The paste according to claim 1, wherein it also contains a vasoconstrictor.

10. The paste according to claim 1, wherein it contains a colorant.

11. The paste according to claim 10, wherein said paste is a paste for application to the oral mucosae and said colorant is a blue colorant.

12. The paste according to claim 1, wherein it also contains a flavouring.

13. The paste according to claim 1, wherein:
it has a rheofluidifying behavior at 23° C.,
it has a viscosity at rest of between 3000 and 4500 Pa·s, measured at 23° C.,
it has a thixotropic behavior, and
it has an adhesive strength of between 0.3 and 0.7 N, measured at 23° C. and ambient humidity.

14. The paste according to claim 1, which has a rheofluidifying behavior characterized by a yield stress of between 110 and 140 Pa, measured at 23° C., and a viscosity of between 60 and 90 Pa·s, measured at a shear rate of 5 s⁻¹ and a temperature of 23° C.

15. A method of stopping the oral mucosae or the skin from bleeding, which comprises the application on said mucosae or skin in need thereof of a paste as defined in claim 1, in efficient amount to form a dressing.

16. The method according to claim 15, wherein said dressing is a hemostatic dressing.
